(19) United States
(12) Patent Application Publication
(10) Pub. No.: US 2023/0087766 A1
(43) Pub. Date: Mar. 23, 2023

(54) METHODS OF REACTIVATING LATENT HUMAN IMMUNODEFICIENCY VIRUS AND RELATED COMPOSITIONS

(71) Applicant: The Regents of the University of California, Oakland, CA (US)

(72) Inventors: Guochun Jiang, Davis, CA (US); Satya Dandekar, Davis, CA (US)

(21) Appl. No.: 17/806,236

(22) Filed: Jun. 9, 2022

Related U.S. Application Data
(63) Continuation of application No. 16/805,429, filed on Feb. 28, 2020, now abandoned.
Application No. 16/805,429 is a continuation of application No. PCT/US2018/048879, filed on Aug. 30, 2018.
(60) Provisional application No. 62/552,952, filed on Aug. 31, 2017.

(51) Int. Cl. A61K 38/44; A61P 31/18; A61K 39/42

(52) U.S. Cl. CPC A61K 38/44; A61P 31/18; A61K 39/42; C12Y 103/01008

(57) ABSTRACT

Methods of reactivating latent human immunodeficiency virus (HIV) in one or more cells of a patient infected with HIV are provided. Methods of treating HIV infection and acquired immune deficiency syndrome (HIV/AIDS) in a patient are also provided. The methods can include administering a crotonylation-inducing agent to the patient. The methods can also include administering a crotonylation-inducing agent and one or more additional latency reversal agents (LRAs) to the patient. Pharmaceutical compositions including a crotonylation-inducing agent or pharmaceutical compositions including a crotonylation-inducing agent and one or more additional LRAs are also provided.

### CROSS-REFERENCE TO RELATED APPLICATIONS

This application is a continuation of U.S. patent application Ser. No. 16/805,429, filed on Feb. 28, 2020, which is a continuation of International Patent Application No. PCT/US2018/048879, filed on Aug. 30, 2018, which claims the benefit of U.S. Provisional Application No. 62/552,952, filed on Aug. 31, 2017, the entire contents of each of which are hereby incorporated by reference in their entirety.

### STATEMENT REGARDING FEDERALLY SPONSORED RESEARCH

This invention was made with government support under Grant No. Al-123105 awarded by the National Institutes of Health. The government has certain rights in the invention.

### TECHNICAL FIELD

The present disclosure relates generally to methods of reactivating latent human immunodeficiency virus (HIV) in one or more cells of a patient infected with HIV. The present disclosure also relates to methods of treating HIV infection and acquired immune deficiency syndrome (HIV/AIDS) in a patient. In particular, the methods can include administering a crotonylation-inducing agent to the patient. The methods can also include co-administering a crotonylation-inducing agent and one or more antiretroviral agents and/or one or more additional latency reversal agents (LRAs) to the patient. Pharmaceutical compositions including a crotonylation-inducing agent or pharmaceutical compositions including a crotonylation-inducing agent and one or more antiretroviral agents and/or one or more additional LRAs are also provided.

### BACKGROUND

Immune cells harboring transcriptionally silent HIV in patients under suppressive antiretroviral therapy (ART) are not generally detected by the host immune system and can pose a barrier to HIV eradication (see K. M. Bruner, et al. Trends in Microbiology 23, 192-203 (2015); D. M. Margolis, Current Infectious Disease Reports 16, 392 (2014); J. D. Siliciano, et al. Nature Medicine 9, 727-728 (2003); D. Finzi, et al. Science 278, 1295-1300 (1997); J. K. Wong, et al. Science 278, 1291-1295 (1997); and T. W. Chun, et al. Proceedings of the National Academy of Sciences of the United States of America 94, 13193-13197 (1997)). HIV transcription is highly regulated and is responsive to several cell signaling pathways. Transcriptional activation of latent HIV has been reported using LRAs that activate protein kinase C/NF-KB and pTEFb signaling (see G. Jiang, et al. PLoS Pathogens 11, e1005066 (2015)). It has been recognized that epigenetic regulation of histone tails at HIV Long Terminal Repeat (LTR) is involved in the establishment of HIV latency (see S. Hakre, et al. Current Opinion in HIV and AIDS 6, 19-24 (2011)); accordingly, histone deacetylase (HDAC) inhibitors or histone methyltransferase inhibitors can reactivate HIV through reprogramming histone tails at HIV LTR and have been assessed as LRAs in human clinical trials (see G. Jiang, et al. Journal of Virology 81, 10914-10923 (2007); I. du Chene, et al. The EMBO Journal 26, 424-435 (2007); J. Friedman, et al. Journal of Virology 85, 9078-9089 (2011); N. M. Archin, et al. Nature 487, 482-485 (2012); T. A. Rasmussen, et al. The Lancet. HIV 1, e13-21 (2014); 0. S. Sogaard, et al. PLoS Pathogens 11, e1005142 (2015)). While HDAC inhibitors can reactivate latent HIV, their potency is low compared to other LRAs (see G. Jiang, et al. PLoS Pathogens 11, e1005066 (2015); G. Jiang, et al. AIDS 28, 1555-1566 (2014); and G. Jiang, et al. AIDS Research and Human Retroviruses 31, 4-12 (2015)). It is possible that additional unknown epigenetic barriers may exist that limit efficient HIV reactivation.

Lysine crotonylation is a recently discovered histone post-translational modification that regulates differential mammalian gene expression (see M. Tan, et al. Cell 146, 1016-1028 (2011) and B. R. Sabari, et al. Molecular Cell 58, 203-215 (2015)). Recent studies showed that addition of sodium crotonate (also referred to herein as “Na-Cro”) to cells increased intracellular levels of crotonyl-CoA and induced histone crotonylation at gene promotor sites (see B. R. Sabari, et al. Molecular Cell 58, 203-215 (2015) and B. R. Sabari, et al. Nature Reviews Molecular Cell Biology 18, 90-101 (2017)). Furthermore, MOF has been shown to possess histone crotonyltransferase activity (see X. Liu, et al. Cell Discovery 3, 17016 (2017), which is hereby incorporated by reference in its entirety). Without being bound by any one specific theory, the concentration or ratio of acetyl-CoA and crotonyl-CoA at the histone tails may play a role in acyl modification and efficient gene transcription. Similar to histone acetylation marks, “readers,” “erasers,” and “writers” of histone crotonylation have been reported (see Y. Li, et al. Transcription, 8, 9-14 (2016); F. H. Andrews, et al. Nature Chemical Biology 12, 396-398 (2016); and X. Bao, et al. eLife 3, (2014)). However, it is not known whether histone crotonylation is involved in HIV transcription and whether it interacts with or influences other histone modifications at the HIV LTR for efficient HIV transcription.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments disclosed herein will become more fully apparent from the following description and appended claims, taken in conjunction with the accompanying drawings.

FIG. 1 is a series of graphs showing reactivation of latent HIV by crotonyl-CoA produced by sodium crotonate addition in HIV latency models. Panel A illustrates that Na-Cro addition reactivated the latent HIV in the resting CD4+ T cells from HIV-infected individuals under ART. Virus expression was measured using Quantitative Viral Outgrowth Assay (QVOA). Panels B-D illustrate that Na-Cro addition reactivated HIV transcription in J-Lat A1 (see panel B) or U1 cells (see panels C and D) as measured by RT-qPCR (see panel C) or by p24 ELISA (see panel D). Panel E illustrates that reactivation of latent HIV was induced using epigenetic modifiers. J-Lat Al Cells were treated with 30 mM Na-Cro, 500 nM suberoylanilide hydroxamic acid (SAHA), 2 μM GSK343, or 5 μM AZA-dc. HIV expression was measured by RT-qPCR. **, p<0.01; ***, p<0.001 compared with control.

FIG. 2 is a series of images and graphs showing that histone crotonylation by sodium crotonate addition re-programs histone tails at the HIV LTR. Panel A illustrates that Na-Cro addition increased global expression of H3K4Cro, H3K4Ac, and H3K18Ac while decreasing H3K27Me3. Panel B illustrates expression of transcription factors after Na-Cro addition in J-Lat Al cells. Panels C-E illustrate that Na-Cro addition re-programmed histone tails at the HIV LTR. Chromatin immunoprecipitation (ChIP) assay was performed by using anti-H3K4Cr, anti-H3K4Ac, and anti-H3K27Me3 antibodies after J-Lat Al cells were treated with 30 mM Na-Cro for 4 hours. PCR primers were specific for the HIV LTR region.

FIG. 3 is a series of graphs showing that suppression of acetyl-CoA synthetase 2 (ACSS2) enzyme disrupts crotonyl-CoA induced reactivation of latent HIV. Panel A illustrates that Na-Cro addition to J-Lat Al cells increased expression of ACSS2 enzyme. **, p<0.01 compared with control treatment. Panel B illustrates expression of ACSS2 after ACSS2 siRNA knockdown in TZM-bl HIV reporter cells. **, p<0.01 compared with control siRNA. Panel C illustrates knockdown of ACSS2 decreased Na-Cro-driven HIV transcription. *, p<0.05 compared with control siRNA. Panels D and E illustrate that pretreatment with ACSS2 inhibitor, AR12, dampens reactivation of latent HIV (see panel D) by inhibition of histone crotonylation (see panel E) at the HIV LTR. **, p<0.01 and ***, p<0.001, compared with control treatment. ##, p<0.01 and ###, p<0.001 compared with Na-Cro treatment.

FIG. 4 is a series of graphs showing a synergistic reactivation of latent HIV by the histone crotonylation in combination with other LRAs. Panel A illustrates that the J-Lat Al cells were treated with LRAs individually or in combination and evaluated for HIV reactivation by RT-qPCR. *, p<0.05; **, p<0.01. Panel B illustrates that the J-Lat Al cells were pre-treated with 250 nM of SAHA then 40 mM of Na-Cro was added over night. The HIV reactivation was measured by real-time PCR. **, p<0.01 compared with control. Panels C and D illustrate that the effect of Na-Cro, individually or in combination, was tested on reactivation of latent HIV in J-Lat A1 cells (see panel C) or U1 cells (see panel D). *, p<0.05; **, p<0.01 compared with control. #, p<0.05, ##, p<0.01 compared with PEP005.

FIG. 5 is a series of graphs and a diagram showing that histone crotonylation modifier and PEP005 synergistically reactivate latent HIV in CD4+ T cells from HIV-infected individuals under suppressive ART. Panels A-C illustrate that primary CD4+ T cells were treated with 100 ng/ml PMA 2 plus 2 μg/ml ionomycin, 12 nM PEP005, 40 mM Na-Cro, or 12 nM PEP005 combined with 40 mM Na-Cro for 6 hours. Viral transcription from total RNA was analyzed by RT-ddPCR with primers targeting initiation (TAR region) (see panel A), elongation (long LTR) (see panel B), or full transcription (poly A region) (see panel C) of the HIV genome. Panel D illustrates a working model of epigenetic regulation of HIV latency/transcription by histone crotonylation.

FIG. 6 is a graph depicting that Na-Cro is not toxic to immune cells. An increased concentration of Na-Cro was added in the J-Lat A1 cells, and cellular viability was evaluated by flow cytometry of live/dead staining.

FIG. 7 is a series of graphs showing that induction of de-crotonylation of histone tail at HIV LTR is independent of SIRTs. Panel A illustrates that J-Lat Al cells were pre-treated with either 50 μM RSV or 1 mM NAM, followed by 30 mM Na-Cro addition to reactivate latent HIV expression. Untreated controls were compared with positive controls involving individual treatment of Na-Cro, RSV, or NAM. *, p<0.05; **, p<0.01 compared with control treatment. #, p<0.05 compared with Na-Cro treatment. Panel B illustrates that gene expression of sirt1 or sirt3 was measured by RT-qPCR in J-Lat Al cells treated with either 25 μM RSV, 50 μM RSV, or 1 mM NAM. *, p<0.05 and **, p<0.01 compared with control treatment. Panels C and D illustrate that histone crotonylation induces binding of SIRT3 and SIRT1 at the HIV LTR. J-Lat Al cells were treated with 30 mM Na-Cro for 4 hours and analyzed by ChIP assays using anti-SIRT1 or anti-SIRT3 antibodies. *, p<0.05 compared with control treatment.

FIG. 8 is a graph depicting pretreatment with Na-Cro enhanced SAHA-reactivation of latent HIV in U1 cells. The U1 cells were pre-treated with 40 mM of Na-Cro then 500 nM SAHA was added, or the cells were treated with 500 nM of SAHA first followed with addition of 40 mM of Na-Cro. After overnight, the cells were collected and the HIV reactivation was measured by real-time PCR. ***, p<0.001, compared with SAHA treatment alone.

FIG. 9 is a series of graphs showing that induction of histone crotonylation at HIV LTR does not enhance immune activation or expression of immune checkpoint marker. PBMCs from healthy HIV-negative donors (n=5) were treated with 100 ng/ml PMA 2 plus 2 μg/ml ionomycin, 30 mM Na-Cro overnight, and analyzed by flow cytometry for the expression of CD4, CD8, HLA-DR, CD69, or PD-1 in CD3+ T cells. Percentage of cells expressing immune activation markers was presented for CD3+ cells (see panel A), CD3+CD4+ T cells (see panel B), and CD3+ CD8+ T cells (see panel C). ***, p<0.001, compared with control after 24 or 72 hour treatment.

FIG. 10 illustrates that SIV infection is regulated by histone crotonylation.

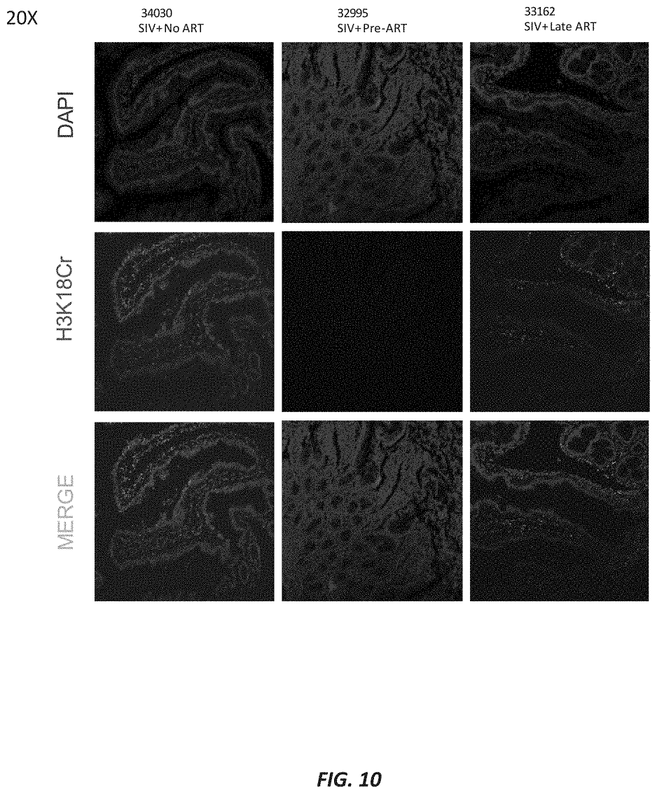

### DETAILED DESCRIPTION

The present disclosure relates to methods of reactivating latent HIV in one or more cells of a patient infected with HIV. The present disclosure also relates to methods of treating HIV/AIDS in a patient. The methods can include administering a crotonylation-inducing agent to the patient. The methods can also include co-administering a crotonylation-inducing agent and one or more additional LRAs to the patient receiving antiretroviral agents. Pharmaceutical compositions including a crotonylation-inducing agent or pharmaceutical compositions including a crotonylation-inducing agent and one or more antiretroviral agents and/or one or more additional LRAs are also disclosed.

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

A “therapeutically effective amount” includes an amount or quantity effective, at dosages and for periods of time necessary, to achieve the desired therapeutic or prophylactic result.

The term “administering,” as used herein, includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering an effective amount of the compounds described herein for reactivating latent HIV infection.

The term “co-administer,” as used herein, indicates that a first compound is administered at the same time, just prior to, or just after the administration of a second compound, e.g., the sequential or simultaneous administration of a crotonylation-inducing agent and one or more LRAs. For example, two or more compounds can be co-administered by administering a pharmaceutical composition adapted for oral administration that contains the two or more compounds. As another example, two or more compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g., sequentially or simultaneously, via intravenous or intramuscular injection.

The terms “antiretroviral therapy” or “ART,” as used herein, include administering one or more compounds simultaneously or sequentially to reduce or suppress the replication of a retrovirus (e.g., HIV) within an infected individual. This can be achieved, for example, by administering various classes of antiretroviral compounds to an infected individual that inhibit various steps of the replication cycle. These classes of compounds include, but are not limited to, fusion inhibitors, nucleoside reverse transcriptase inhibitors, integrase inhibitors, protease inhibitors, and combinations thereof. For example, ART can be used to treat viral infections caused by HIV. One non-limiting goal of this therapy in HIV, for example, is to decrease and/or eliminate viral levels and increase the number of CD4+ T cells in HIV-infected individuals.

The term “latent HIV,” as used herein, includes a state of the HIV life cycle in which the HIV genome has integrated into the chromosomal DNA of the infected cell, but replication of the genome and HIV proliferation in the infected cell is dormant. In some instances, the dormant viral replication impedes the treatment of HIV infection with ART because ART targets various stages of the HIV replication cycle. For example, latent HIV virus cannot be treated using ART, and is a known problem inhibiting the functionality of antiretroviral treatment techniques.

The term “reactivating a latent HIV,” as used here, includes administering a compound or combination of compounds to induce a latent HIV-infected cell into an active replication and proliferation state.

The terms “latency reversal agent” or “LRA,” as used herein, include a compound or combination of compounds capable of reactivating a latent virus. For example, the terms include a single compound that is capable of inducing reactivation of a latent HIV. As another example, the terms also include a combination of two or more compounds that are capable of synergistically or additively inducing reactivation of a latent HIV. LRAs are useful for treating a variety of latent viral infections. LRAs include, but are not limited to, any compound belonging to a class selected from a histone methyltransferase inhibitor, an HDAC inhibitor, a DNA methyltransferase inhibitor, a protein kinase C (PKC) agonist, a crotonylation-inducing agent, and combinations thereof.

The term “crotonylation-inducing agent,” as used herein, includes a compound or combination of compounds capable to inducing crotonylation. For example, the term includes a compound or combination of compounds capable of modifying a protein by the introduction of one or more crotonyl groups. As another example, the term includes a compound or combination of compounds capable of modifying one or more lysine residues in a histone by the introduction of one or more crotonyl groups. Crotonylation-inducing agents include, but are not limited to, sodium crotonate, crotonyl-coenzyme A, agents that can activate and/or induce crotonyl-CoA converting enzyme ACSS2, agents that can activate and/or induce p300/CBP and/or MOF, and combinations thereof.

The term “pharmaceutically acceptable,” as used herein, includes a substance which does not interfere with the effectiveness or the biological activity of the active ingredients and which is not toxic to the hosts in the amounts used, and which hosts may be either humans or animals to which it is to be administered.

The terms “synergistic” or “synergistically,” as used herein, include an enhanced therapeutic benefit or effect where a combination of two or more compounds produces a result that exceeds their expected additive effect. For example, synergistic LRAs enhance latent viral reactivation to an extent that exceeds their expected additive effect.

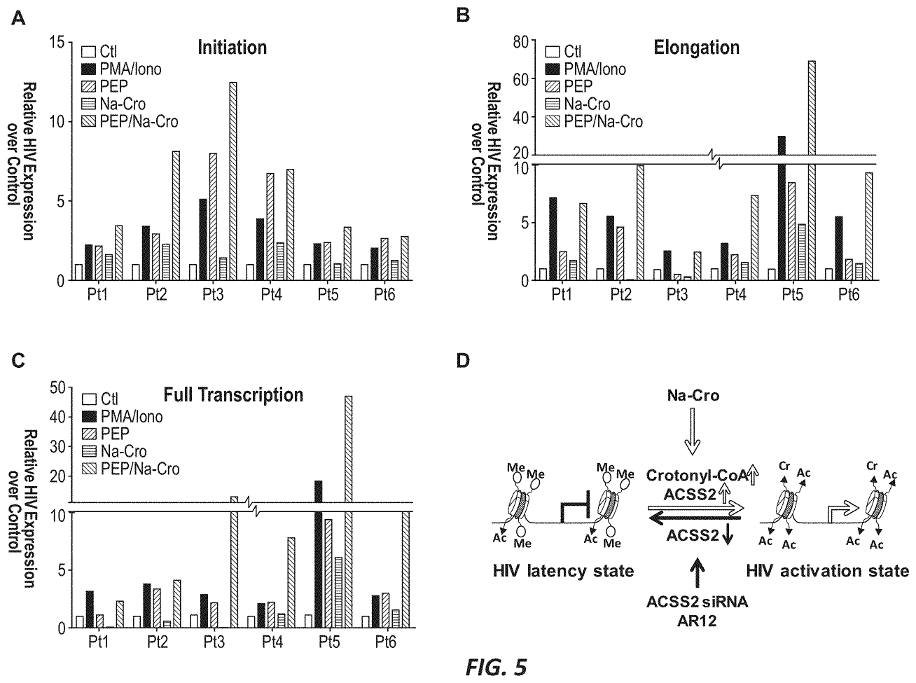

Herein, novel epigenetic modification—histone crotonylation—is described that can regulate HIV transcription and govern viral latency. It was found that histone crotonylation disrupted the latent state of HIV infection after Na-Cro induction of ACSS2 (see FIG. 5, panel D). Latent HIV reservoirs are established during early stages of viral infection in the host and utilize multiple molecular mechanisms (see S. Dandekar, et al. Current HIV/AIDS Reports 4, 10-15 (2007); A. A. Lackner, et al. Cold Spring Harbor Perspectives in Medicine 2, a007005 (2012); M. Somsouk, et al. AIDS 29, 43-51 (2015); L. A. Hirao, et al. PLoS Pathogens 10, e1004311 (2014); and J. B. Whitney, et al. Nature 512, 74-77 (2014)). The HIV LTR can be integrated mostly in active regions of the host genome (see S. Hakre, et al. Current Opinion in HIV and AIDS 6, 19-24 (2011)). Therefore, a quiescent chromatin environment and interactions of cellular and viral transcriptional regulators with the HIV transcriptional machinery may be useful for inducing and maintaining HIV latency (see G. Jiang, A. et al. Journal of Virology 81, 10914-10923 (2007) and M. Tyagi, et al. The EMBO Journal 26, 4985-4995 (2007)). Latent HIV is reactivated by interference with chromatin modifications as evidenced by efficacy of HDAC inhibitors or EZH2 inhibitors (see G. Jiang, et al. AIDS Research and Human Retroviruses 31, 4-12 (2015)). However, the level of HIV reactivation achieved has been relatively modest in T cell cultures in vitro and in CD4+ T cells from HIV-infected patients ex vivo (see G. Jiang, et al. PLoS Pathogens 11, e1005066 (2015); G. Jiang, et al. AIDS 28, 1555-1566 (2014); Z. Klase, et al. iPLoS Pathogens 10, e1003997 (2014); D. Pandelo Jose, et al. Virology 462-463, 328-339 (2014); and S. Bouchat, et al. EMBO Molecular Medicine 8, 117-138 (2016)). Without being bound by any one particular theory, it may be possible that HIV latency is regulated by additional histone modifications that have yet to be discovered. As provided herein, histone crotonylation can be an epigenetic mark regulating HIV latency.

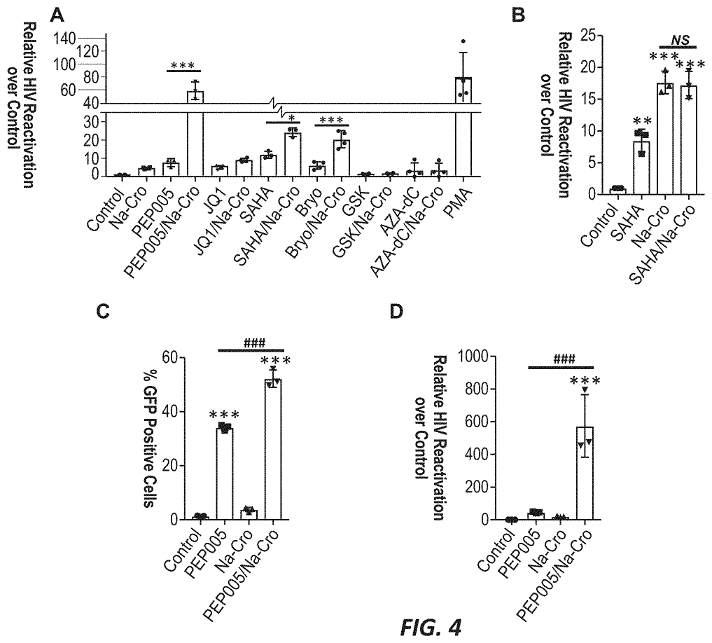

It was found that there was a synergistic reactivation of latent HIV with a combination of histone crotonylation and PKC agonist PEP005, SAHA, or JQ1 in T cell cultures in vitro and/or CD4+ T cells from HIV-infected patients ex vivo. Na-Cro treatment did not induce immune activation or modulate levels of immune checkpoint protein PD-1 on CD4+ T cells or CD8+ T cells. Instead, Na-Cro addition led to a decrease in the expression of the immune activation marker HLA-DR. In the presence of Na-Cro, the ability of the HDAC inhibitor SAHA to reactivate latent HIV was significantly enhanced, indicating that efficient reactivation of latent HIV by an HDAC inhibitor may require crotonylation of histone tails at HIV LTR (see FIG. 4). Inhibition of histone crotonylation by suppressing crotonyl-CoA converting enzyme ACSS2 resulted in dampening of latent HIV reactivation by inhibition of histone acetylation and histone crotonylation. These data support a cross-talk among multiple histone modifications at HIV LTR, indicating a potential role of histone de-crotonylation in HIV latency. It has been shown that acetylation of Tat by SIRT1 is required for an efficient HIV transcription by modulating interaction of Tat with TAR (see S. Pagans, et al. PLoS biology 3, e41 (2005)). This may indicate that some other enzymes are involved in de-crotonylating histone tails at the HIV LTR, such as class I HDACs (see X. Bao, et al. eLife 3, (2014); J. L. Feldman, et al. The Journal of Biological Chemistry 288, 31350-31356 (2013); and W. Wei, et al. Cell Research, (2017)).

A first aspect of the disclosure relates to methods of reactivating one or more latent viruses in one or more cells of a patient infected with the one or more viruses. The methods can include administering one or more crotonylation-inducing agents to the patient.

In some embodiments, the virus can be selected from the group consisting of HIV, cytomegalovirus (CMV), adenoviruses, papovaviruses, herpesviruses, varicella-zoster virus, Epstein-Barr virus, pox viruses, vaccinia virus, hepatitis B virus, rhinoviruses, hepatitis A virus, poliovirus, rubella virus, hepatitis C virus, arboviruses, rabies virus, influenza viruses A and B, measles virus, mumps virus, and HTLV I and II. In particular embodiments, the virus is selected from the group consisting of an HIV, a cytomegalovirus (CMV), and an adenovirus.

In certain embodiments, the methods can include reactivating latent HIV in one or more cells of a patient infected with HIV. In some embodiments, the one or more agents can reactivate the latent HIV in the one or more cells of the patient. The crotonylation-inducing agent may be selected from at least one of sodium crotonate, crotonyl-coenzyme A (crotonyl-CoA), agents that can activate and/or induce crotonyl-CoA converting enzyme ACSS2, agents that can activate and/or induce p300/CBP and/or MOF, and/or another suitable crotonylation-inducing agent.

In certain embodiments, the method can include co-administration of the one or more crotonylation-inducing agents with one or more additional LRAs. The additional LRA may be selected from at least one of an HDAC inhibitor, a PKC agonist, and/or another suitable LRA. In some embodiments, the method can include co-administration of the one or more crotonylation-inducing agents with one or more anti-HIV antibodies. An effect of the one or more crotonylation-inducing agents in combination with the one or more additional LRAs and/or the one or more anti-HIV antibodies may be a synergistic effect.

In various embodiments, the method may further include administering one or more HDAC inhibitors to the patient. The HDAC inhibitor may be selected from at least one of suberanilohydroxamic acid (SAHA), suberoyl bis-hydroxamic acid (SBHA), trichostatin A (TSA), scriptaid, oxamflatin, givinostat (ITF2357), belinostat (PXD101), droxinostat, romidepsin, panobinostat, CG05/CG06, valproic acid (VPA), sodium butyrate, apicidin, and/or another suitable HDAC inhibitor. For example, in some embodiments, the method may include co-administering a crotonylation-inducing agent and an HDAC inhibitor to the patient. The one or more crotonylation-inducing agents may be administered just prior to, simultaneously with, or just after the administration of the one or more HDAC inhibitors.

In some embodiments, the method may further include administering one or more PKC agonists to the patient. The PKC agonist may be selected from at least one of ingenol-3-angelate (PEP005), 12-deoxyphorbol-13-acetate (prostratin), bryostatin-1, an analog thereof, and/or another suitable PKC agonist. For example, in some embodiments, the method may include co-administering a crotonylation-inducing agent and a PKC agonist to the patient. As another example, the method may include co-administering a crotonylation-inducing agent, an HDAC inhibitor, and a PKC agonist to the patient. The one or more crotonylation-inducing agents may be administered just prior to, simultaneously with, or just after the administration of the one or more PKC agonists and/or the one or more HDAC inhibitors.

In certain embodiments, the method may further include administering one or more anti-HIV antibodies to the patient. For example, in various embodiments, the method may include co-administering a crotonylation-inducing agent and an anti-HIV antibody to the patient. As another example, the method may include co-administering a crotonylation-inducing agent and any combination of an HDAC inhibitor, a PKC agonist, and/or an anti-HIV antibody to the patient. The one or more crotonylation-inducing agents may be administered just prior to, simultaneously with, or just after the administration of the one or more PKC agonists, the one or more HDAC inhibitors, and/or the one or more anti-HIV antibodies.

In various embodiments, the patient is human. In some embodiments, the patient is being treated with ART, e.g., highly active antiretroviral therapy (HAART), suppressive ART, etc. The method may further include administering ART to the patient or continuing to administer ART to the patient.

The one or more crotonylation-inducing agents may be administered with at least one of a pharmaceutically acceptable carrier, an excipient, and/or a diluent. Likewise, the HDAC inhibitor, the PKC agonist, and/or the anti-HIV antibody may be administered with at least one of a pharmaceutically acceptable carrier, an excipient, and/or a diluent.

Another aspect of the disclosure relates to methods of treating HIV/AIDS in a patient. The methods may include administering one or more crotonylation-inducing agents to the patient having HIV/AIDS. The methods may also include administering a therapeutically effective amount of one or more crotonylation-inducing agents to the patient having HIV/AIDS.

In some embodiments, the administration of the one or more crotonylation-inducing agents may reactivate a latent HIV in the patient. The method can also include co-administration of the one or more crotonylation-inducing agents with one or more additional LRAs. As stated above, the additional LRA may be selected from at least one of an HDAC inhibitor, a PKC agonist, and/or another suitable LRA. In certain embodiments, the method can include co-administration of the one or more crotonylation-inducing agents with one or more anti-HIV antibodies. An effect of the one or more crotonylation-inducing agents in combination with the one or more additional LRAs and/or the one or more anti-HIV antibodies may be a synergistic effect.

In various embodiments, the methods may further include administering one or more HDAC inhibitors to the patient. In some embodiments, the methods may further include administering one or more PKC agonists to the patient. In certain embodiments, the methods may further include administering one or more anti-HIV antibodies to the patient. Furthermore, the one or more crotonylation-inducing agents may be administered just prior to, simultaneously with, or just after the administration of the one or more PKC agonists, the one or more HDAC inhibitors, and/or the one or more anti-HIV antibodies.

In various embodiments, the patient is human. In some embodiments, the patient is being treated with ART. The methods may further include administering ART to the patient or continuing to administer ART to the patient. Additionally, the crotonylation-inducing agent, the HDAC inhibitor, the PKC agonist, and/or the anti-HIV antibody may be administered with at least one of a pharmaceutically acceptable carrier, an excipient, and/or a diluent.

Another aspect of the disclosure relates to pharmaceutical compositions for treating a patient infected with HIV. The pharmaceutical composition may include a crotonylation-inducing agent.

The pharmaceutical composition may also include one or more crotonylation-inducing agents and any combination of HDAC inhibitor(s), PKC agonist(s), and/or anti-HIV antibody(s). Furthermore, the pharmaceutical composition may include at least one of a pharmaceutically acceptable carrier, an excipient, and/or a diluent. The pharmaceutical composition may, or may be configured to, reactivate a latent HIV in one or more cells of a patient infected with HIV and/or treat HIV/AIDS in a patient.

Another aspect of the disclosure relates to suppressing one or more “erasers” of crotonylation to reactivate one or more latent viruses in a patient infected with the virus. In some embodiments, suppressing or inhibiting the one or more “erasers” can reactivate latent viruses (e.g., HIV) in the patient.

As will be understood by one of ordinary skill in the art, each embodiment disclosed herein can comprise, consist essentially of, or consist of its particular stated element, step, ingredient, or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components, and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

The compounds described herein are useful in the manufacture of a pharmaceutical composition or a medicament for reactivating latent HIV or latent HIV reservoirs in an infected subject. In certain aspects, a pharmaceutical composition or medicament can be administered to a subject for the treatment of an HIV infection that leads to the reduction or elimination of the virus from latent reservoirs in infected resting cells such as central and/or transitional memory CD4+ T cells. In certain other aspects, a pharmaceutical composition or medicament can be administered to a subject for the treatment of a viral infection such as an HIV infection that leads to the reactivation of virally infected cells from latent reservoirs and activation of the subject's immune system to combat and kill the recently reactivated virally infected cells.

Pharmaceutical compositions or medicaments for use in the present disclosure can be formulated by standard techniques or methods well-known in the art of pharmacy using one or more physiologically acceptable carriers or excipients. Suitable pharmaceutical carriers are described herein and in, e.g., “Remington's Pharmaceutical Sciences” by E. W. Martin. Compounds and agents of the present disclosure and their physiologically acceptable salts and solvates can be formulated for administration by any suitable route, including, but not limited to, orally, topically, nasally, rectally, pulmonary, parenterally (e.g., intravenously, subcutaneously, intramuscularly, etc.), and combinations thereof. In some embodiments, the therapeutic agent is dissolved in a liquid, for example, water. The most suitable route of administration in any given case will depend in part on the nature, severity, and optionally, the stage of the viral infection. Co-administration of a plurality or combination of compounds may be by the same or different route of administration or together in the same pharmaceutical formulation.

Examples of pharmaceutically acceptable carriers include sterile liquids, such as water and oils, including those of petroleum, animal, vegetable or synthetic origin, such as peanut oil, soybean oil, mineral oil, and sesame oil. Aqueous carriers, including water, are typical carriers for pharmaceutical compositions prepared for intravenous administration. As further examples, saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. The composition, if desired, can also contain wetting or emulsifying agents, or pH buffering agents.

The pharmaceutical compositions described herein can be formulated using any of the active compounds described herein, including any pharmaceutically acceptable salts, esters, isomers, or solvates thereof. In certain embodiments, the pharmaceutical compositions described herein include an active compound as described herein, and in alternative embodiments, the pharmaceutical compositions include two or more active compounds according to the present description. The amount of the one or more active compounds included in the pharmaceutical composition will vary, depending upon, for example, the nature and activity of the active compound(s), the nature and composition of the dosage form, and the desired dose to be administered to a subject.

It should be understood, however, that a specific dosage and treatment regime for any particular subject or disease state will depend upon a variety of factors, including the age, body weight, general health, gender, diet, time of administration, nature of active compound(s), rate of excretion, drug combination, the judgment of the treating physician, and the severity of the particular disease being treated. Moreover, determination of the amount of a pharmaceutical composition to be administered to a subject will depend upon, among other factors, the amount and specific activity of the active compound(s) included in the pharmaceutical composition and the use or incorporation of additional therapeutic or prophylactic agents or treatment regimes. Determination of therapeutically effective dosages may be based on animal model studies and is typically guided by determining effective dosages and administration protocols that significantly reduce the occurrence or severity of disease in model subjects.

A non-limiting range for a therapeutically effective amount of the active compounds described herein is from about 0.001 mg/kg to about 100 mg/kg body weight per day. For example, pharmaceutical compositions according to the present description can be prepared and administered such that the amount of active compound according to the present description administered to a subject is selected from between about 0.001 mg/kg and about 50 mg/kg, between about 0.01 mg/kg and about 20 mg/kg, between about 0.1 and about 10 mg/kg, and between about 0.1 mg/kg and about 5 mg/kg body weight per day.

### EXAMPLES

The following examples are illustrative of disclosed methods and compositions. In light of this disclosure, those of skill in the art will recognize that variations of these examples and other examples of the disclosed methods and compositions would be possible without undue experimentation.

### Example 1

### Induction of Histone Crotonylation Reactivates HIV from Latency.

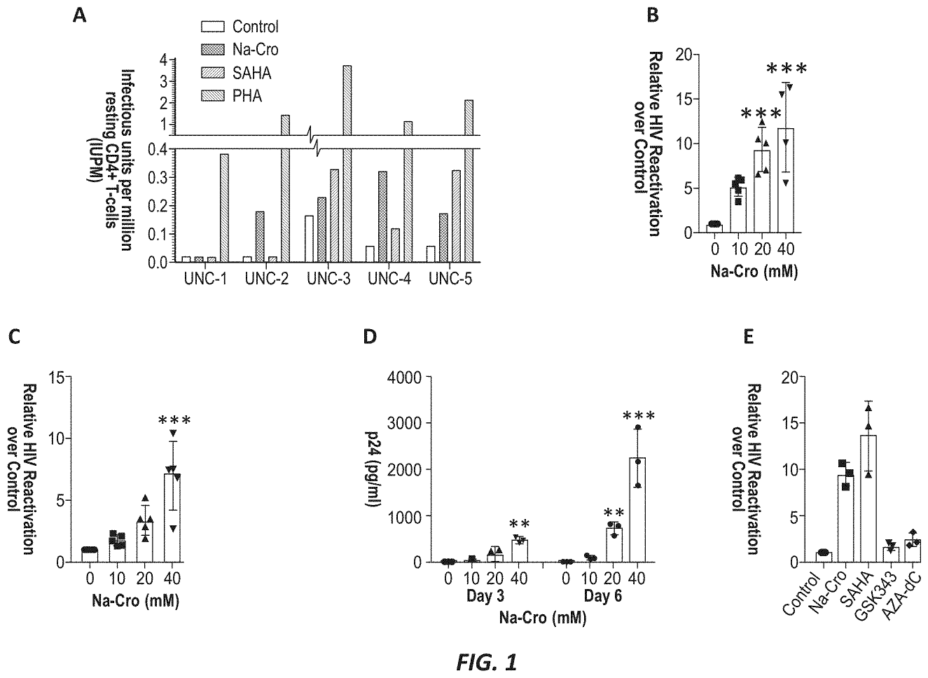

It was tested whether the histone crotonylation inducer, sodium crotonate, can reactivate latent HIV reservoirs from a patient under ART. Peripheral blood samples were obtained from 5 HIV-infected individuals with undetectable plasma viral loads and higher CD4+ T cell numbers (see Table 1). One million resting CD4+ T cells were treated with IL-2, PHA+IL-2, or IL-2+Na-Cro, and HIV p24 production was measured by QVOA using p24 ELISA as a measure of HIV reactivation. In four of the five participants, replication-competent HIV was recovered following exposure to Na-Cro, suggesting histone crotonylation allows the reversal of HIV latency ex vivo (see FIG. 1, panel A). Similar to Na-Cro, SAHA was able to flush out the latent HIV in these patient cells except for one patient (UNC-1).

TABLE 1
Characteristics of the HIV-1-infected Participants

| | | | | | | Years |
|---|---|---|---|---|---|---|
| | | | | CD4 | | under |
| Pt ID | Age | Sex | VL | count | ART | ART |
| Pt#001 | 62 | M | <20 | 788 | Epzicom and Neviripine | >10 |
| Pt#002 | 59 | M | <20 | 682 | Atripa | >10 |
| Pt#003 | 58 | M | <20 | 502 | Triumeq | >17 |
| Pt#004 | 59 | M | <20 | 885 | Triumeq | >8 |
| Pt#005 | 46 | F | 0 | 465 | Stribild | >12 |
| Pt#006 | 54 | M | <20 | 267 | Truvada and dolutegravir | >15 |
| UNC-1 | 32 | M | <40 | 666 | Odefsey | >3 |
| UNC-2 | 59 | M | <20 | 733 | Atazanavir, Ritonavir, | >6 |
| | | | | | FTC, Tenofovir | |
| UNC-3 | 28 | M | <40 | 701 | Darunavir, Ritonavir, | >5 |
| | | | | | Truvada | |
| UNC-4 | 45 | F | <20 | 1050 | Atripla | >6 |
| UNC-5 | 25 | M | <40 | 535 | Triumeq | >1 |

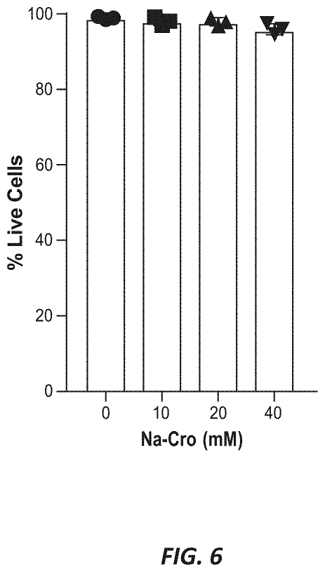

The analysis of histone crotonylation for latent HIV reactivation was expanded to two well-characterized cell-based HIV latency models in vitro, including J-Lat A1 nd U1 cells. Induction of histone crotonylation by the addition of sodium crotonate to these cell cultures resulted in the induction of HIV LTR-driven transcription in a dose-dependent manner. Previous studies in tumor cell lines showed that addition of sodium crotonate to cells increased cellular levels of crotonyl-CoA, which in turn caused histone crotonylation and induced gene transcription (see B. R. Sabari, et al. Molecular Cell 58, 203-215 (2015)). J-Lat Al cells harbor a single copy of HIV provirus with a GFP reporter gene under the control of an HIV-1 LTR promoter. Addition of Na-Cro at 40 mM concentration increased HIV transcription by 12-fold compared to untreated controls (see FIG. 1, panel B). Minimal cell toxicity was observed in the presence of Na-Cro ranging from 10 mM to 40 mM (see FIG. 6). In U1 cells (a U937-derived promonocyte cell line that contains two copies of complete HIV genome with defective Tat gene), addition of Na-Cro at 40 mM induced a 6-fold increase in viral transcription compared to untreated controls (see FIG. 1, panel C). Similarly, addition of 20 mM and 40 mM of Na-Cro yielded over 500 pg/mL and 2000 pg/mL of p24 antigen at day 6 (see FIG. 1, panel D). Multiple epigenetic modifications of histone tails have been shown to regulate HIV transcription. Inhibiting some of these chromatin repressors by LRAs can reactivate latent HIV. It was found that while histone methyltransferase (EZH2) inhibitor GSK343 or DNA methyltransferase inhibitor AZA-dC minimally reactivated latent HIV, histone crotonylation inducer Na-Cro and HDAC inhibitor SAHA had comparable capacity to reactivate latent HIV in J-Lat Al cells (see FIG. 1, panel E). Collectively, these data demonstrate that induction of histone crotonylation can be effective in disrupting HIV latency across multiple HIV latency models ex vivo and in vitro.

### Example 2

### Increased Crotonyl-Coa Concentrations Re-Program Chromatin Structure Through Histone Crotonylation Regulated by ACSS2 Enzyme.

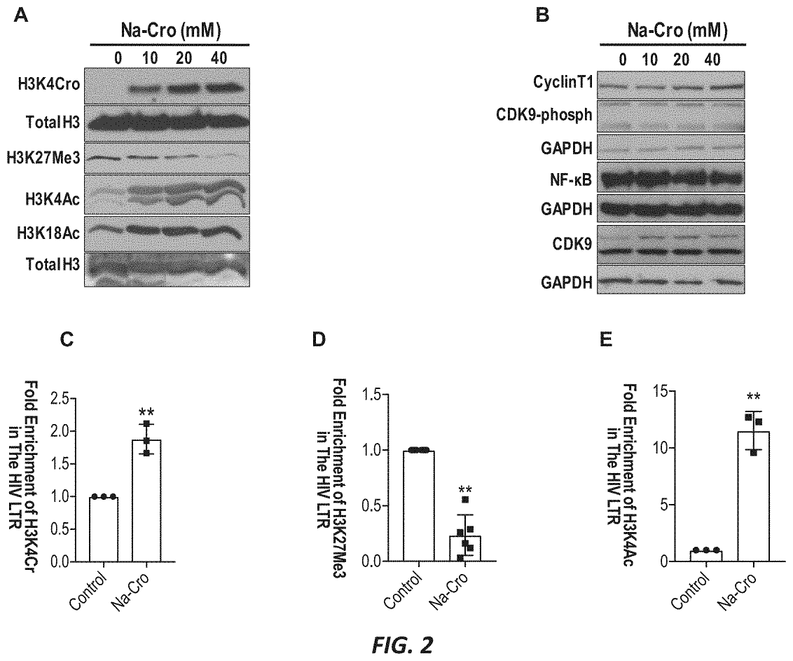

To determine the molecular mechanism of crotonyl CoA-induced reactivation of latent HIV, the HIV LTR associated histone modifications in cell line models of HIV latency were examined. Several histone modifications were searched for in Na-Cro-treated T cells and expression of key components of transcription factors involving HIV transcription and viral latency were assessed. Addition of Na-Cro not only increased H3K4 crotonylation but also H3K4 acetylation and H3K18 acetylation (see B. R. Sabari, et al. Molecular Cell 58, 203-215 (2015)). Addition of Na-Cro also markedly decreased H3K27 trimethylation. Changes in histone crotonylation and acetylation were detectable even at low Na-Cro concentrations (see FIG. 2, panel A). These data indicated that crotonyl-CoA production following Na-Cro exposure is able to globally re-program histone tails in immune cells; however, it did not alter transcription factors that are involved in HIV transcription except a minor induction of CDK9 protein (see FIG. 2, panel B). Addition of Na-Cro increased crotonylation and acetylation at H3K4 associated with decreased histone tri-methylation at H3K27 (see FIG. 2, panels C-E).

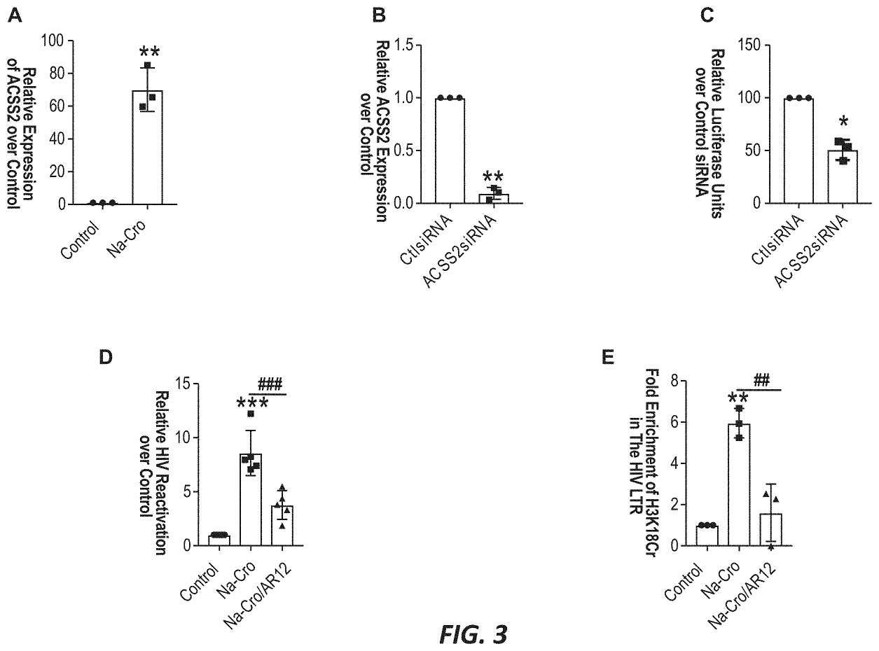

By charging crotonate with CoA-SH, the acyl-CoA synthetase 2 enzyme (ACSS2) enzyme converts crotonate (provided by Na-Cro) into crotonyl-CoA to form crotonyl-CoA, which is utilized by the p300 enzyme to add acyl groups to histones. Notably, Na-Cro treatment induced a 70-fold increase in ACSS2 expression (see FIG. 3, panel A). To define the role of ACSS2, ACSS2 expression was knocked down by using an ACSS2 specific siRNA in the HIV-LTR reporter cell line TZM-bl and it was found that Na-Cro-induced transcription of HIV was significantly reduced (˜70%) compared to the control knockdown (see FIG. 3, panels B and C). These data show that Na-Cro-mediated transcription of HIV was predominantly driven by ACSS2.

To further establish the role of histone crotonylation in the establishment of HIV latency, the activity of ACSS2 was pharmacologically suppressed using AR12, a known ACSS2 inhibitor, and the downstream effects on HIV transcription were investigated (see K. Koselny, et al. ACS Infectious Diseases 2, 268-280 (2016)). Cells treated with Na-Cro had 8-fold higher HIV reactivation compared to the control. In contrast, addition of ACSS2 inhibitor, AR12, resulted in substantial decrease in latent HIV reactivation (>60%). Thus, pretreatment with AR12 diminished the effects of crotonyl-CoA in disrupting HIV latency (see FIG. 3, panel D). Pharmacologic inhibition of the ACSS2 enzyme markedly dampened histone crotonylation at HIV LTR (see FIG. 3, panel E). Collectively, the data show that ACSS2-driven crotonylation is involved in remodeling of the histone tails and contributes to HIV reactivation. This identifies the mechanism of the establishment of HIV latency through inhibition of ACSS2 and lack of histone crotonylation at HIV LTR.

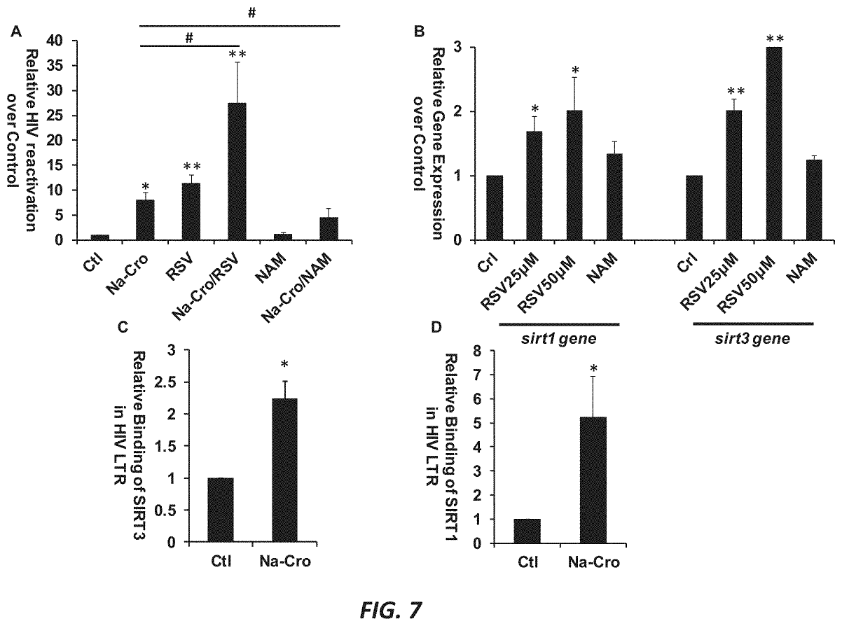

Previous studies identified SIRT1 and SIRT3 proteins from the Sirtuin (SIRTs) family as capable of erasing crotonylation marks from lysine residues in HeLa S3 cell lines (see X. Bao, et al. eLife 3, (2014)). It was determined whether these proteins regulated de-crotonylation of histones at the HIV LTR, thereby silencing the viral transcription. Using compounds with known activating or inhibitory activity for SIRT proteins, the role of SIRT1 and SIRT3 in crotonylation-mediated reactivation of latent HIV was examined. Resveratrol (RSV) is a chemical activator of SIRT1 while nicotinamide (NAM) is a competitive inhibitor of SIRT3. It was found that RSV pretreatment augmented crotonyl-CoA-mediated latent HIV reactivation by 27-fold compared to crotonyl-CoA alone. Pretreatment with NAM reduced viral reactivation to less than 4-fold (see FIG. 7, panel A). Further analysis of the gene expression of sirt1 and sirt3 showed that RSV did in fact increase the expression of sirt1 and sirt3 by two to three-fold (see FIG. 7, panel B). Analysis of ChIP assays on the cells treated the Na-Cro showed that induced SIRT1 and SIRT3 efficiently bound to the HIV LTR at higher levels than controls (see FIG. 7, panels C and D). Therefore, without being bound by any one particular theory, the data did not support an essential role of SIRT1 or SIRT3 as de-crotonylases at HIV LTR and suggest that HIV latency may be regulated independent of sirtuins.

### Example 3

### Crotonyl-CoA Reactivates HIV Synergistically with Other Latency Reversal Agents.

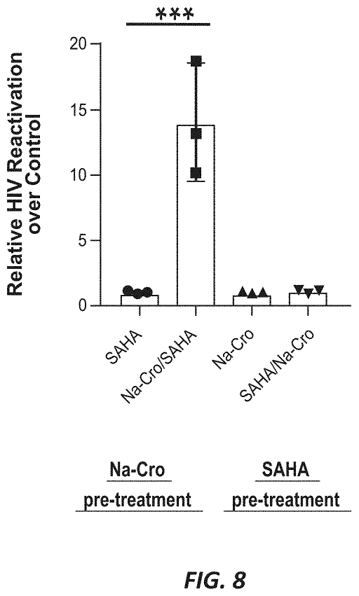

Multiple molecular signaling pathways are involved in the establishment and maintenance of HIV latency. Therefore, an efficient reactivation of latent HIV in the “shock-and-kill” approach may require a combination of LRAs targeting different latency mechanisms. It was determined whether a histone crotonylation mechanism was synergistic with other LRAs having different mechanisms of action for HIV reactivation. In combination treatments, J-Lat A1 cells were pretreated with Na-Cro first and other LRAs were then added. The data showed that Na-Cro was able to synergistically reactivate HIV when combined with PEP005, JQ1, bryostatin-1, or SAHA, respectively, in combination with Na-Cro, reactivation of latent HIV was increased by 8-fold, 1.8-fold, 3.3 fold, and 2.0 fold as compared to single treatment of PEP005, JQ1, bryostatin-1, or SAHA (see FIG. 4, panel A). PEP005 and bryostatin-1 have been shown to reactivate HIV through the PKC/NF-κB pathway (see G. Jiang, et al. PLoS pathogens 11, e1005066 (2015) and L. Diaz, et al. Scientific Reports 5, 12442 (2015)). Synergy in reactivation of latent HIV was found for a combination treatment with Na-Cro and JQ1 (see C. Banerjee, et al. Journal of Leukocyte Biology 92, 1147-1154 (2012)). SAHA is an HDAC inhibitor that can activate HIV expression through chromatin remodeling by inhibiting histone deacetylation (see N. M. Archin, et al. Nature 487, 482-485 (2012)). In contrast, a combination of Na-Cro with histone methyltransferase inhibitor GSK343 or DNA methyltransferase inhibitor AZA-dC did not show any synergistic effect on HIV reactivation in J-Lat Al cell lines. Prior addition of Na-Cro followed by SAHA treatment demonstrated a synergistic effect on HIV reactivation (see FIG. 4, panel A) while a pretreatment with SAHA and followed by Na-Cro addition abolished the combination effect in both J-Lat A1 cells and U1 cells of HIV latency models (see FIG. 4, panel B and FIG. 8). Because a combination of Na-Cro and PEP005 displayed the most potent effect in latent HIV reactivation, the magnitude of HIV LTR reactivation was further assessed and supporting evidence was found for increased viral reactivation by flow cytometric analysis and by RT-qPCR evaluation (see FIG. 4, panel C). Similar synergistic increase in HIV reactivation was also identified in U1 monocytic cells (see FIG. 4, panel D).

### Example 4

### Histone Crotonylation Suppresses Expression of Immune Activation Marker HLA-DR but not of Immune Checkpoint Marker PD-1.

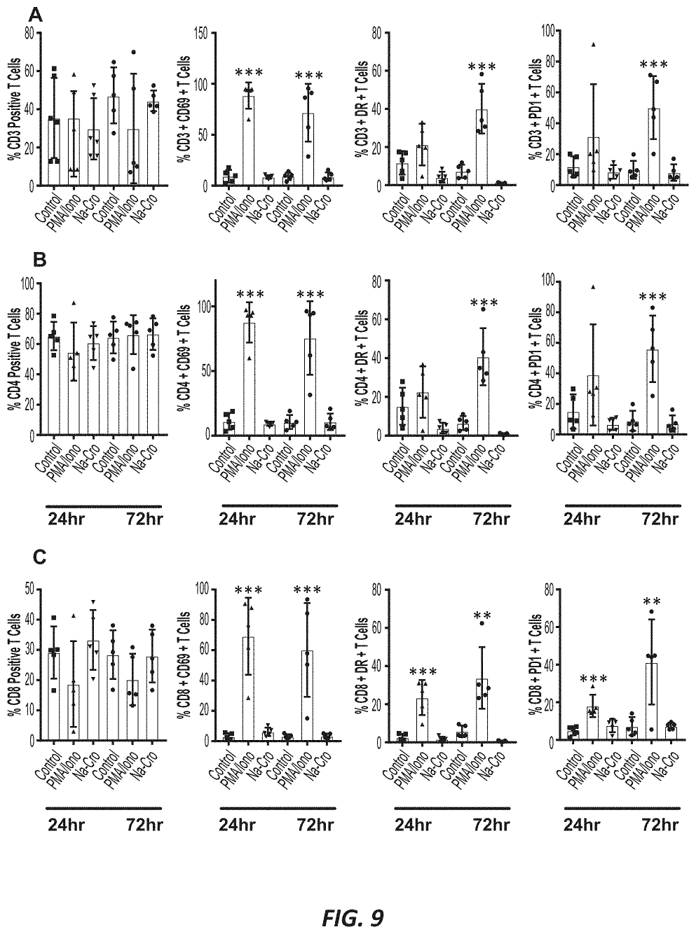

It was examined whether induction of histone crotonylation in T cells impacted the level of immune activation in these cells. Peripheral blood mononuclear cells from HIV-negative healthy donors (n=5) were treated with PMA/Ionomycin or Na-Cro for 24 hours or 72 hours. Changes in the immune cell status were examined by measuring the levels of HLA-DR, CD69, and PD-1 in the T cell subsets by flow cytometric analysis (see FIG. 9). In CD3+ T cells, crotonylation suppressed HLA-DR expression at 72 hours post-treatment while PMA/ionomycin significantly induced HLA-DR expression at 24 hours and 72 hours of treatment (see FIG. 9, panel A). Na-Cro addition did not change expression of the immune checkpoint marker PD-1 in CD3+ T cells. In CD4+ or CD8+ T cells, addition of Na-Cro inhibited HLA-DR expression at 24 and 72 hours of treatment. Similarly, Na-Cro did not modulate PD-1 expression in these cells (see FIG. 9, panels B and C). These findings suggest that histone crotonylation does not cause any changes in immune activation of T cells. Instead, it is associated with the suppression of immune activation, which may have a beneficial effect on patient immune function.

### Example 5

### Induction of Histone Crotonylation in Combination with Other LRAs Disrupts HIV Latency in Primary CD4+ T Cells from HIV Infected Patients Under Suppressive ART.

In HIV latency cell culture models in vitro, it was found that Na-Cro displayed a greatest increase in HIV reactivation while in combination with PKC agonist PEP005 as compared to other LRAs (see FIG. 4, panel A). To validate this finding in the ex vivo primary CD4+ T cells from patients the peripheral blood samples from 6 HIV-infected patients under suppressive ART were obtained. They had undetectable plasma viral load (<20 copies/ml plasma) and >400 CD4+ T cell number per ml of blood (465-885 cells/ml) (see Table 1). Primary CD4+ T cells were treated with PMA/Ionomycin, PEP005, Na-Cro, or PEP005 in combination with Na-Cro for 6 hours. HIV transcription following reactivation was measured by digital droplet PCR (ddPCR) targeting TAR region (initiation), long LTR region (elongation), or Poly A region (full transcription) of HIV genome (see G. Jiang, et al. PLoS pathogens 11, e1005066 (2015); P. Kaiser, et al. Journal of virological methods 242, 1-8 (2017); and L. Shan, et al. Journal of Virology 87, 6521-6525 (2013)). Concordant with the findings from resting CD4+ T cells using QVOA ex vivo and HIV latent models in vitro, histone crotonylation readily induced initiation and elongation of HIV transcripts from primary CD4+ T cells of all 6 patients, as well as full-length transcription of HIV in samples from 4 of the 6 patients (see FIG. 5, panels A-C). An addition of PEP005 induced the initiation of HIV transcription in CD4+ T cells from all six patients while supporting the elongation of long viral transcripts in 5 of 6 patient samples. Similar to previous reports, PEP005 alone induced full-length transcription of HIV in 5 of 6 patient samples (see G. Jiang, et al. PLoS Pathogens 11, e1005066 (2015)). These data show that Na-Cro can induce HIV transcription in primary CD4+ T cells from HIV-infected patients under suppressive ART. In primary CD4+ T cells from HIV-infected patients, combination of Na-Cro with PEP005 induced greater reactivation of latent HIV than either agent alone (see FIG. 5, panels A-C), even higher than by PMA/Ionomycin treatment, indicating a higher potency when combination strategy is applied. These findings are in agreement with the data from HIV latency cell culture models in vitro. Collectively, without being bound by any one particular theory, the data may suggest that Na-Cro not only is able to reactivate latent HIV in primary CD4+ T cells from HIV-infected patients under ART but also is synergistic with PEP005, a LRA targeting PKC/NF-KB signaling, in its reactivation activity.

### Example 6

### SIV Infection is Regulated by Histone Crotonylation

SIV infection induces epigenetic modification of histone crotonylation in the gut mucosa. Initiation of early anti-retroviral therapy suppresses viral replication and this coincides with decreased histone crotonylation. Intestinal tissues from SIV-infected rhesus macaques were processed for the analysis of immunohistochemistry (IHC) to detect histone 3 crotonylation at lysine 18 (H3K18Kc). IHC was performed using 4% paraformaldehyde (PFA) fixed, paraffin embedded tissues. Five micrometer sections were dehydrated and antigen retrieval (DAKO™) was performed at 95 ° C. for 30 minutes. Tissues were blocked with 1% Fc blockers (MILTENYI BIOTEC™) and 15% donkey serum (JACKSON IMMUNORESEARCH™ Laboratories Inc.) for 1 hour and 30 minutes, then incubated with primary antibody (H3K18Cr, rabbit pAB, PTM BIOLABS™) overnight at 4° C. in dark, followed by secondary antibody (donkey anti-rabbit, ALEXA FLUOR® 488, JACKSON IMMUNORESEARCH™ Laboratories Inc.) for 1 hour at room temperature. Samples were stained with DAPI and Sudan Black, immersed in mounting solution (DAKO™) covered by glass slips and sealed. Imaging was performed with the ZEISS™ 780 confocal microscope using 20× air objectives at optical resolution settings. 34030: intestinal tissue from animal infected with SIV for 21 weeks and is therapy-naïve; 32995: intestinal tissue from animal infected with SIV for 33 weeks but initiated ART at 1 week post-infection; 33162: intestinal tissue from animal infected for 38 weeks but initiated ART about 6 weeks after infection.

The following materials and methods were used in the Examples above:

Cell culture: J-Lat Al cells (derived from Jurkat cells harboring HIV LTR-Tat-GFP gene) and U1 cells (promonocytic cell line harboring one copy of HIV genome with defective tat gene) were obtained from NIH AIDS Reagent Program and cultured at 37° C. with 5% CO₂ in RPM11640 medium containing 10% FBS and 1% penicillin-streptomycin as previously described (see G. Jiang, et al. PLoS Pathogens 11, e1005066 (2015)).

Latency Reversal Agents: For reactivation of latent HIV, cells were treated with sodium crotonate (SIGMA-ALDRICH®' St. Louis, Mo.), PEP005 (TOCRIS® Bioscience, Bristol, UK), SAHA (SANTA CRUZ BIOTECHNOLOGY™, Santa Cruz, Calif.), GSK343 (SIGMA-ALDRICH®), or AZA-Dc (SANTA CRUZ BIOTECHNOLOGY™) for 4, 18, or 24 hours. A combination treatment involved pretreatment of cells with sodium crotonate for 4 hours, followed by treatment with PEP005, Brystatin-1 (CALBIOCHEM®, Billerica, Mass.), JQ1 (BIOVISION™, Milpitas, Calif.), SAHA, GSK343, or AZA-Dc for 18 hours. For inhibiting ACSS2 and crotonylation, cells were pretreated with ACSS2 inhibitor, AR12 (SELLECK CHEMICALS™, Houston, Tex.), for 30 minutes, followed by Na-Cro treatment for 4 or 18 hours.

Gene Knockdown by ACSS2 siRNA: 1×10⁵ of TZM-bl HIV transcription/replication reporter cells were seeded in the 12-well plate, and then the cells were transfected with ACSS2 siRNA (M-010396) or non-targeting control siRNA (M-006526) (DHARMACON™, UK) twice. After treatment with 40 mM Na-Cro overnight, the cells were collected for luciferase assay of HIV transcription or lysed for RT-qPCR of ACSS2 gene expression.

Western Blot Analysis: Western blot analysis was performed by using one million cells treated with LRAs for 6 or 18 hours. Whole cell lysates were prepared using RIPA buffer. Nuclear Extracts were prepared using EPIQUIK™ Total Histone Extraction Kit (EPIGENTEK™, Farmingdale N.Y.). Antibodies (CELL SIGNALING TECHNOLOGY®, Boston, Mass.) for Cyclin T1, CDK9, CDK9-P, NF-kB, GAPDH, and total H3 were used to detect protein expression under different drug conditions. Antibodies for H3K4Cro (PTM BIOLABS™, Chicago, Ill.), H3K27Me3 (CELL SIGNALING TECHNOLOGY®), H3K4Ac (CELL SIGNALING TECHNOLOGY®), and H3K18Ac (CELL SIGNALING TECHNOLOGY®) were used to measure histone modifications.

Chromatin Immunoprecipitation (ChIP): J-Lat Al cells (2.5 to 5 million) were utilized in ChIP assays with anti-H3K18Ac, anti-H3K4Cr, anti-H3K27Me3, anti-H3K4Ac, anti-SIRT3 (CELL SIGNALING TECHNOLOGY®), and anti-SIRT1 (CELL SIGNALING TECHNOLOGY®) antibodies. ChIP assays were performed using SIMPLECHIP® Kit from CELL SIGNALING TECHNOLOGY® as per the manufacturer's recommendations. Samples were cross-linked using formaldehyde and 150-1000 bp fragments were made using Micrococcal Nuclease digestion and 7 cycles of 30 seconds on/30 seconds off sonication. Antibodies were used to probe respective proteins. Purification of DNA was performed using the SIMPLECHIP® Kit Protocol. Purified ChIP DNA was used in SYBR® Green qPCR using ChIP primers specific for HIV LTR.

Real-Time PCR Analysis: HIV gene expression was measured using real-time PCR in J-Lat Al cells treated with LRAs. Total nucleic acid was extracted using RNEASY™ Kit, genomic DNA digested using DNase I and total RNA reverse transcribed using Superscript III to synthesize cDNA. HIV gene expression was determined by quantitative real-time PCR (ABI ViiA 7 detector) using the following primer/probe set: for J-Lat A1 cells, primer 1: 5′-GGAGCGACCATCTTCTTCA-3′ (SEQ ID NO:1), primer 2: 5′-AGGGTGTCGCCCTCGAA-3′ (SEQ ID NO:2), probe 5′-FAM CTACAAGACCC GCGCCGAGGTG TAMRA-3′ (SEQ ID NO:3).

Primary CD4+ T Cell Isolation, Treatment, and Digital Droplet PCR Assays: Peripheral blood samples were obtained from HIV-infected individuals (age ranging from 46-62 years old) on suppressive ART for >8 years (n=6). The plasma viral loads were below the detective level (<20 copies/ml plasma) and the average CD4+ T cell number was 664.4±180.5 cells/ml. The primary CD4+ T cells were isolated using the EASYSEP™ kit (STEMCELL™ Technologies Inc., Vancouver, BC, Canada) as previously described (see G. Jiang, et al. PLoS pathogens 11, el 005066 (2015) and G. Jiang, et al. AIDS 28, 1555-1566 (2014)). The purified CD4+ T cells were plated at a density of 0.5-1×10⁶ cells and treated with Control, 200 ng/ml PMA plus 2 μM lonomycin, 12 nM PEP005, 40 mM Na-Cro, or 12 nM PEP005 plus 40 mM Na-Cro for 6 hours. Cell pellets were collected for RNA isolation. Initiation, elongation, or full transcription of HIV was analyzed with ddPCR assays as reported before (see G. Jiang, et al. PLoS pathogens 11, e1005066 (2015); P. Kaiser, et al. Journal of virological methods 242, 1-8 (2017); and L. Shan, et al. Journal of virology 87, 6521-6525 (2013)).

Quantitative Viral Outgrowth Assay: Peripheral blood mononuclear cells (PBMC) were obtained from HIV-infected individuals on suppressive ART (n=5) by continuous-flow leukopheresis. Isolation of resting CD4+ T cells and quantification of replication competent virus was performed as previously described (see S. Dandekar, et al. Current HIV/AIDS reports 4, 10-15 (2007)). Briefly, approximately 34-50 million resting CD4⁺ T cells per each treatment condition were plated in replicate limiting dilutions of 2.5 million (18 cultures), 0.5 million (6 cultures), and 0.1 million (6 cultures) cells per well and stimulated for 24 hours with either: 1) PHA (REMEL™, THERMO™ Scientific) and a 5-fold excess of allogeneic irradiated PBMCs from a seronegative donor, and 60 U/ml IL-2 for 24 hours; 2) 40 mM Na-Cro, or 3) 5 U/ml IL-2 as unstimulated control. Cultures were washed and co-cultivated with CD8+ T cell-depleted PBMCs that were obtained from selected HIV sero-negative donors previously screened for adequate CCRS expression. Culture supernatants were harvested on days 15 and 19 and assayed for virus production by p24 antigen capture ELISA (ABL). Cultures were scored as positive if p24 was detected at day 15 and was increased in concentration at day 19. The number of resting CD4+ T cells in infected units per million (IUPM) was estimated by a maximum likelihood method (see N. M. Archin, et al. AIDS 23, 1799-1806 (2009)).

Statistical Analysis: Means and standard errors (SE) were calculated for all data points from at least 3 independent experiments. Statistical significance was determined using the two-way Student t-test, where p value <0.05 considered significant. Coefficient correlation was analyzed by using MICROSOFT™ EXCEL™

Certain embodiments of this disclosure are described herein, including the best mode known to the inventors for carrying out the disclosure. Of course, variations on these described embodiments will become apparent to those of ordinary skill in the art upon reading the foregoing description. The applicants expect skilled artisans to employ such variations as appropriate, and the applicants intend for the various embodiments of the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents and printed publications throughout this specification. Each of the above-cited references and printed publications are individually incorporated herein by reference in their entirety.

It is to be understood that the embodiments of the present disclosure are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure.

It will be apparent to those having skill in the art that many changes may be made to the details of the above-described embodiments without departing from the underlying principles of the disclosure. The scope of the present invention should, therefore, be determined only by the following claims.

1. A method of reactivating a latent human immunodeficiency virus (HIV) in one or more cells of a patient infected with HIV, the method comprising administering a crotonylation-inducing agent to the patient to reactivate the latent HIV in the one or more cells of the patient.

2. The method of claim 1, wherein the crotonylation-inducing agent is selected from at least one of sodium crotonate, crotonyl-coenzyme A (crotonyl-CoA), an agent that can activate crotonyl-CoA converting enzyme ACSS2, or an agent that can activate p300/CBP and/or MOF.

3. The method of claim 1, further comprising administering a histone deacetylase (HDAC) inhibitor to the patient.

4. The method of claim 3, wherein the HDAC inhibitor is selected from at least one of suberanilohydroxamic acid (SAHA), suberoyl bis-hydroxamic acid (SBHA), trichostatin A (TSA), scriptaid, oxamflatin, givinostat (ITF2357), belinostat (PXD101), droxinostat, romidepsin, panobinostat, CG05/CG06, valproic acid (VPA), sodium butyrate, or apicidin.

5. The method of claim 1, further comprising administering a protein kinase C (PKC) agonist to the patient.

6. The method of claim 5, wherein the PKC agonist is selected from at least one of ingenol-3-angelate (PEP005), 12-deoxyphorbol-13-acetate (prostratin), bryostatin-1, or an analog thereof.

7. The method of claim 1, further comprising administering an anti-HIV antibody to the patient.

8. The method of claim 1, wherein the patient is being treated with a suppressive antiretroviral therapy (ART).

9. The method of claim 1, further comprising administering suppressive ART to the patient.

10. The method of claim 1, wherein the crotonylation-inducing agent is administered with at least one of a pharmaceutically acceptable carrier, an excipient, or a diluent.

11. A method of treating human immunodeficiency virus infection and acquired immune deficiency syndrome (HIV/AIDS) in a patient, the method comprising administering a crotonylation-inducing agent to the patient to reactivate a latent human immunodeficiency virus (HIV) in the patient.

12. The method of claim 11, wherein the crotonylation-inducing agent is selected from at least one of sodium crotonate, crotonyl-coenzyme A (crotonyl-CoA), an agent that can activate crotonyl-CoA converting enzyme ACSS2, or an agent that can activate p300/CBP and/or MOF.

13. The method of claim 11, further comprising administering a histone deacetylase (HDAC) inhibitor to the patient.

14. The method of claim 13, wherein the HDAC inhibitor is selected from at least one of suberanilohydroxamic acid (SAHA), suberoyl bis-hydroxamic acid (SBHA), trichostatin A (TSA), scriptaid, oxamflatin, givinostat (ITF2357), belinostat (PXD101), droxinostat, romidepsin, panobinostat, CG05/CG06, valproic acid (VPA), sodium butyrate, or apicidin.

15. The method of claim 11, further comprising administering a protein kinase C (PKC) agonist to the patient.

16. The method of claim 15, wherein the PKC agonist is selected from at least one of ingenol-3-angelate (PEP005), 12-deoxyphorbol-13-acetate (prostratin), bryostatin-1, or an analog thereof.

17. The method of claim 11, further comprising administering an anti-HIV antibody to the patient.

18. The method of claim 11, wherein the patient is being treated with a suppressive antiretroviral therapy (ART).

19. The method of claim 11, further comprising administering suppressive ART to the patient.

20. The method of claim 11, wherein the crotonylation-inducing agent is administered with at least one of a pharmaceutically acceptable carrier, an excipient, or a diluent.

21. A pharmaceutical composition for treating a patient infected with a human immunodeficiency virus (HIV), the pharmaceutical composition comprising a crotonylation-inducing agent.

22. The pharmaceutical composition of claim 21, wherein the crotonylation-inducing agent is selected from at least one of sodium crotonate, crotonyl-coenzyme A (crotonyl-CoA), an agent that can activate crotonyl-CoA converting enzyme ACSS2, or an agent that can activate p300/CBP and/or MOF.

23. The pharmaceutical composition of claim 21, further comprising a histone deacetylase (HDAC) inhibitor.

24. The pharmaceutical composition of claim 23, wherein the HDAC inhibitor is selected from at least one of suberanilohydroxamic acid (SAHA), suberoyl bis-hydroxamic acid (SBHA), trichostatin A (TSA), scriptaid, oxamflatin, givinostat (ITF2357), belinostat (PXD101), droxinostat, romidepsin, panobinostat, CG05/CG06, valproic acid (VPA), sodium butyrate, or apicidin.

25. The pharmaceutical composition of claim 21, further comprising a protein kinase C (PKC) agonist.

26. The pharmaceutical composition of claim 25, wherein the PKC agonist is selected from at least one of ingenol-3-angelate (PEP005), 12-deoxyphorbol-13-acetate (prostratin), bryostatin-1, or an analog thereof.

27. The pharmaceutical composition of claim 21, further comprising an anti-HIV antibody.

28. The pharmaceutical composition of claim 21, further comprising at least one of a pharmaceutically acceptable carrier, an excipient, and a diluent.

29. A method of reactivating a latent virus in one or more cells of a patient infected with the virus, the method comprising administering a crotonylation-inducing agent to the patient to reactivate the latent virus in the one or more cells of the patient.

30. The method of claim 29, wherein the crotonylation-inducing agent is selected from at least one of sodium crotonate, crotonyl-coenzyme A (crotonyl-CoA), an agent that can activate crotonyl-CoA converting enzyme ACSS2, or an agent that can activate p300/CBP and/or MOF.

31. The method of claim 29, further comprising administering a histone deacetylase (HDAC) inhibitor to the patient.

32. The method of claim 31, wherein the HDAC inhibitor is selected from at least one of suberanilohydroxamic acid (SAHA), suberoyl bis-hydroxamic acid (SBHA), trichostatin A (TSA), scriptaid, oxamflatin, givinostat (ITF2357), belinostat (PXD101), droxinostat, romidepsin, panobinostat, CG05/CG06, valproic acid (VPA), sodium butyrate, or apicidin.

33. The method of claim 29, further comprising administering a protein kinase C (PKC) agonist to the patient.

34. The method of claim 33, wherein the PKC agonist is selected from at least one of ingenol-3-angelate (PEP005), 12-deoxyphorbol-13-acetate (prostratin), bryostatin-1, or an analog thereof.

35. The method of any one of claim 29, further comprising administering an antibody that is specific to the virus to the patient.

36. The method of claim 29, wherein the patient is being treated with a suppressive antiviral therapy.

37. The method of claim 29, further comprising administering suppressive antiviral therapy to the patient.

38. The method of claim 29, wherein the crotonylation-inducing agent is administered with at least one of a pharmaceutically acceptable carrier, an excipient, or a diluent.

39. A pharmaceutical composition for treating a patient infected with a virus, the pharmaceutical composition comprising a crotonylation-inducing agent.

40. The pharmaceutical composition of claim 39, wherein the crotonylation-inducing agent is selected from at least one of sodium crotonate, crotonyl-coenzyme A (crotonyl-CoA), an agent that can activate crotonyl-CoA converting enzyme ACSS2, or an agent that can activate p300/CBP and/or MOF.

41. The pharmaceutical composition of claim 39, further comprising a histone deacetylase (HDAC) inhibitor.

42. The pharmaceutical composition of claim 41, wherein the HDAC inhibitor is selected from at least one of suberanilohydroxamic acid (SAHA), suberoyl bis-hydroxamic acid (SBHA), trichostatin A (TSA), scriptaid, oxamflatin, givinostat (ITF2357), belinostat (PXD101), droxinostat, romidepsin, panobinostat, CG05/CG06, valproic acid (VPA), sodium butyrate, or apicidin.

43. The pharmaceutical composition of claim 39, further comprising a protein kinase C (PKC) agonist.

44. The pharmaceutical composition of claim 43, wherein the PKC agonist is selected from at least one of ingenol-3-angelate (PEP005), 12-deoxyphorbol-13-acetate (prostratin), bryostatin-1, or an analog thereof.

45. The pharmaceutical composition of claim 39, further comprising an antibody specific to the virus.

46. The pharmaceutical composition of claim 39, further comprising at least one of a pharmaceutically acceptable carrier, an excipient, and a diluent.
